(19) United States
(12) Patent Application Publication
(10) Pub. No.: US 2010/0285139 A1
(43) Pub. Date: Nov. 11, 2010

(54) PHARMACEUTICAL COMPOSITION FOR THE PROPHYLAXIS AND/OR SYMPTOMATIC TREATMENT OF CYSTIC FIBROSIS

(75) Inventor: Erich Gulbins, Essen (DE)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: CYCNAD GMBH & CO., KG, Essen (DE)

(21) Appl. No.: 12/809,620

(22) PCT Filed: Dec. 22, 2008
(86) PCT No.: PCT/EP2008/010996

§ 371 Date: Jul. 20, 2010

(30) Foreign Application Priority Data
Dec. 21, 2007 (DE) 10 2007 063 535.6

(51) Int. Cl. A61K 9/14; A61K 39/395; A61P 31/04; A61K 31/137; A61K 31/55; A61K 31/382; A61K 31/138; A61K 31/44; A61K 31/135; A61M 11/00

(52) U.S. Cl. 424/489; 424/130.1; 514/654; 514/217; 514/437; 514/651; 514/356; 514/657; 128/200.14

(57) ABSTRACT

A pharmaceutical composition for prophylaxis and/or treatment of infections and/or infectious diseases occurring in cystic fibrosis includes at least one antidepressant; at least one dispersant; and at least one pharmaceutically tolerable carrier material.

### RELATED APPLICATIONS

This is a §371 of International Application No. PCT/EP2008/010996, with an international filing date of Dec. 22, 2008 (WO 2009/083211 A2, published Jul. 9, 2009), which is based on German Patent Application No. 10 2007 063 535.6, filed Dec. 21, 2007, the subject matter of which is incorporated by reference.

### TECHNICAL FIELD

This disclosure relates to a pharmaceutical composition for the prophylaxis and/or symptomatic treatment of cystic fibrosis, an appropriate administration aid, appropriate kits, a cartridge, methods for the prophylaxis and/or treatment of cystic fibrosis, and the appropriate use of the pharmaceutical composition.

### BACKGROUND

Infections with pseudomonads, in particular Pseudomonas aeruginosa, are a serious clinical problem, in particular in the case of patients with immunosuppression, severe trauma and burns. Thus the lethality of, for example, bacteremias caused by Pseudomonas aeruginosa is about 20%. If pneumonia or a surgical complication is additionally present, the mortality rate in this type of bacteremia can even climb to 30 to 50%. The clinical significance of Pseudomonas aeruginosa is in particular illustrated by the fact that this pseudomonad type plays a crucial role in about 40% of all cases of death of patients with respiration-induced pneumonia.

Pseudomonas aeruginosa especially has the most important clinical significance in patients with cystic fibrosis (mucoviscidosis). Cystic fibrosis or mucoviscidosis is a genetically caused, autosomally recessive hereditary metabolic disorder. It is based on a mutation of the gene for the chloride ion channel CFTR (Cystic Fibrosis Transmembrane Conductance Regulator). The genetic defect of the CFTR protein leads to various clinical problems, in particular pulmonary and gastrointestinal symptoms. From the clinical point of view, the pulmonary symptoms are especially problematic. Thus, in the course of cystic fibrosis chronic pneumonia with pseudomonads, in particular Pseudomonas aeruginosa, occurs in nearly all patients, which is the main reason for the destruction of the lung and the premature death of these patients. Cystic fibrosis is the most frequent autosomally recessive hereditary disease, at least in the European Union (EU) and the USA. At a rate of one affected child to 2500 births, in the EU alone about 40,000 children and young adults annually develop cystic fibrosis.

In WO 2004/017949 A2, the applicability of various substances for the prophylaxis and/or therapy of certain infectious diseases is described. The use of amitriptyline and imipramine, in particular, for the systemic treatment of viral infections is described there.

To improve the prophylaxis and, in particular, the symptomatic therapy of cystic fibrosis and, in particular, to take into account known problems, we faced the task of making available a pharmaceutical composition which is particularly suitable for the prophylaxis and/or symptomatic treatment of cystic fibrosis. In addition, we faced the task of making available an appropriate administration aid and an appropriate kit.

### SUMMARY

We provide a pharmaceutical composition for prophylaxis and/or treatment of infections and/or infectious diseases occurring in cystic fibrosis including at least one antidepressant, at least one dispersant, and at least one pharmaceutically tolerable carrier material.

We also provide an aerosolized pharmaceutical composition, including particles with an aerodynamic diameter in a range from about 0.1 to 12 μm, where the particles contain at least one pharmaceutically tolerable, inhalable carrier material and at least one antidepressant.

We further provide a kit for prophylaxis and/or treatment of infections and/or infectious diseases occurring in cystic fibrosis, including at least one antidepressant, at least one dispersant and/or at least one pharmaceutically tolerable carrier material.

We still further provide a kit including a device for atomizing a pharmaceutical composition and a cartridge which is connected to the device, and an aerosolizable, inhalable pharmaceutical composition, including at least one dispersant and/or at least one pharmaceutically tolerable carrier material and at least one antidepressant.

We further yet provide a cartridge for use in the intrapulmonary release of active ingredient, including a container with a nozzle opening and an aerosolizable, inhalable pharmaceutical composition, including at least one dispersant and/or at least one pharmaceutically tolerable carrier material and at least one antidepressant.

We also further provide a method for inhibiting acid sphingomyelinase in lungs of a patient, including aerosolizing or atomizing a pharmaceutical composition including at least one dispersant and/or at least one pharmaceutically tolerable carrier material and at least one antidepressant, and inhaling the aerosolized or atomized pharmaceutical composition into the lungs of the patient.

### BRIEF DESCRIPTION OF THE DRAWINGS

The figures schematically show:

FIG. 1 shows the influence of various antidepressants on the activity of acid sphingomyelinase in Cftr-deficient mice in comparison to untreated Cftr-deficient mice and untreated wild-type mice;

FIG. 2 shows the influence of various antidepressants on the cellular ceramide concentration in Cftr-deficient mice in comparison to untreated Cftr-deficient mice and untreated wild-type mice;

FIG. 3 shows the influence of various antidepressants on the susceptibility to infection in Cftr-deficient mice in comparison to untreated Cftr-deficient mice and untreated wild-type mice;

FIG. 4 shows the influence of various antidepressants on the pulmonary concentration of the inflammatory mediator interleukin-1β in Cftr-deficient mice in comparison to untreated Cftr-deficient mice and untreated wild-type mice;

FIG. 5 shows the influence of amintriptyline in various administration forms on the activity of acid sphingomyelinase in Cftr-deficient mice and wild-type mice;

FIG. 6 show the influence of amitriptyline in various administration forms on the cellular ceramide concentration in Cftr-deficient mice and wild-type mice; and

FIG. 7 shows the influence of amitriptyline in various administration forms on the susceptibility to infection in Cftr-deficient mice and wild-type mice.

### DETAILED DESCRIPTION

We provide a pharmaceutical composition or medicament for the prophylaxis and/or symptomatic treatment of cystic fibrosis, in particular, for the prophylaxis and/or treatment of infections and/or infectious diseases occurring in cystic fibrosis, comprising at least one antidepressant and preferably at least one dispersant and/or at least one pharmaceutically tolerable carrier material.

We surprisingly discovered that an accumulation of ceramide occurs in the pulmonary epithelial cells of mice that do not express any Cftr (Cystic fibrosis transmembrane conductance regulator) in the lung. In the case of Cftr, this is the mouse equivalent to CFTR, which is why mice that do not express any Cftr—“Cftr-deficient mice or Cftr-knockout mice”—are suitable model organisms for cystic fibrosis. In other words, in the presence of cystic fibrosis the pulmonary ceramide concentration is markedly increased in comparison to a healthy organism. The increase in the ceramide concentration in the epithelial cells of the lungs is based in particular here on a pH increase (alkalization) in intracellular vesicles. On account of the increase in the intravesicular pH, the ceramide-degrading enzyme acid ceramidase is more strongly inhibited than the ceramide-releasing enzyme acid sphingomyelinase. This inequilibrium with respect to the inhibition of acid ceramidase and acid sphingomyelinase causes an accumulation or enrichment of ceramide in the intracellular vesicles of pulmonary epithelial cells. The ceramide accumulation finally leads to the death of the epithelial cells. If the cells die, cell constituents, in particular DNA, reach the bronchial lumen. Pathogens present in the lung, in particular pseudomonads, can adhere to these cell constituents. Microbial population or colonization of the lung is thereby promoted, whereby the risk in turn increases that the pulmonary symptoms of cystic fibrosis mentioned at the start manifest themselves in the affected patients. Moreover, ceramide leads to a chronic inflammation in the lung of Cftr-deficient mice.

It was interestingly possible to show by Cftr-deficient mice or Cftr-knockout mice that the composition causes a stronger inhibition of acid sphingomyelinase than acid ceramidase on intrapulmonary, preferably inhalative, administration. In addition, it was possible to show that the composition led to an extensive normalization of the ceramide concentration in the pulmonary epithelial cells of the experimental animals. It was also possible to detect a significant reduction of inflammatory reactions of the pulmonary epithelial cells after administration of the composition. With particular advantage, it was moreover possible to show that after administration of the composition the susceptibility to infection of the experimental animals to pulmonary pathogens, in particular Pseudomonas aeruginosa, normalized.

Preferably, the antidepressant is present dispersed in the dispersant. The dispersant is preferably liquid. With particular advantage, the dispersant is an aqueous liquid, in particular a physiological fluid. It is particularly preferred if the dispersant is a solvent or contains such a solvent. Preferred dispersants are selected from the group consisting of water, ethanol, isopropanol and/or dimethyl sulfoxide (DMSO). For example, the dispersant can be a physiological electrolyte, buffer or salt solution, for example saline solution.

The composition is preferably present in the form of a pharmaceutical formulation. The formulation itself can take place in gaseous, liquid, semisolid or solid state. Correspondingly, the composition can be present, for example, as an aerosol, spray, dispersion, gel, paste, ointment, tablet or powder. A liquid, in particular aqueous, formulation is particularly preferred. The composition can be present as a liquid dispersion, in particular, as a suspension or solution. Preferably, the composition is present as an aqueous dispersion, particularly preferably as an aqueous solution.

The composition may be present in the form of an inhalant or inhalation agent. The inhalant or inhalation agent can here be a spray, in particular, a nasal or oral spray, or a dropper solution.

The composition may contain excipients or additives, for example, binders, plasticizers, diluents, glidants, antistatics, antioxidants, adsorption agents, mold-release agents, dispersants, coated tablet lacquer, antifoams, film formers, emulsifiers, extenders, pigments and/or fillers. If present in liquid form, the composition can contain, for example, flavor-improvement additives, preservatives, stabilizers and/or buffers. The additives can be of inorganic and/or organic nature. For example, the additives can be fats, fatty alcohols, fatty acids, waxes, polysaccharides, sugars, proteins, peptides, amino acids, vitamins, trace elements, salts, acids, bases, alcohols, polymers and the like. Suitable sugars are preferably monosaccharides, disaccharides and/or sugar alcohols. Examples of suitable sugars can be selected from the group consisting of glucose, sucrose, lactose, threose, galactose, mannitol and sorbitol. A suitable protein is, for example, human serum albumin. Examples of suitable salts are sodium chloride, potassium chloride, calcium chloride and/or magnesium chloride. Further suitable excipients are selected from the group consisting of cetyltrimethylammonium bromide, BDSA, cholate, deoxycholate, polysorbate 20 and 80, fusidic acid and lipids, in particular, cationic lipids. The composition can furthermore contain one or more additives from the group consisting of solubility-increasing additives, preferably cyclodextrin, hydrophilic additives, preferably mono- or oligosaccharides, absorption-promoting additives, preferably cholate, deoxycholate, fusidic acid or chitosan, cationic surfactants, preferably cetyl-trimethylammonium bromide, viscosity-increasing additives, preferably carboxymethylcellulose, maltodextrin, alginic acid, hyaluronic acid or chondroitin sulfate, a release matrix, for example, based on polyanhydride or polyorthoester, a hydrogel, a particulate, slow-release depot system, preferably based on polylactide-co-glycolide (PLG), a depot foam, starch or systems derived from cellulose. The composition can furthermore contain a lipid-based carrier material, preferably in the form of an emulsion and/or based on liposomes, niosomes or micelles. In addition, the composition can also contain additives which destabilize “bilayer arrangements,” preferably phosphatidylethanolamine. Further possible additives are, for example, fusogenic additives, preferably cholesterol hemisuccinate.

Furthermore, possible pharmaceutically tolerable additives or excipients can be volatile or nonvolatile. Examples of suitable classes of excipients of this type are gaseous, or liquid or solid solvents in the supercritical state. Suitable excipients are therefore in particular selected from the group consisting of water, terpenes, such as, for example, menthol, alcohols, such as, for example, ethanol, propylene glycol, glycerol and other similar alcohols, dimethylformamide, dimethylacetamide, wax, supercritical carbon dioxide, dry ice and mixtures thereof.

The composition can furthermore also contain a propellant, for example, compressed air, nitrogen, hydrofluoroalkanes and the like. In principle, all possible propellants can be used here which are inert to the antidepressant and optionally further pharmaceutically active compounds which are contained in the composition.

As already mentioned, the composition can contain a pharmaceutically tolerable carrier material alternatively or in combination to a dispersant. Suitable carrier materials are described in detail in the literature (Remington's Pharmaceutical Sciences, 16th edition, 1980, Mack Publishing Co., edited by Oslo et al.) The carrier material can be present in the form of particles in the composition, it being possible for the particles themselves to have a non-inhalable particle size. For example, the carrier material can contain particles having a particle size of between 10 and 500 μm, in particular 50 and 200 μm.

If the composition is present as a powder, in particular, a pulverulent formulation, basically all carrier materials suitable for this purpose are possible. Suitable examples are glucose, lactose, lactose monohydrate, sucrose, trehalose, sugar alcohols, such as, for example, mannitol or xylitol, polylactide and/or cyclodextrins.

Possible liquid carrier materials are preferably water, buffer solutions, electrolyte or salt solutions, aqueous dextrose solutions and/or glycol solutions. In addition, the carrier material can also be selected from various oils, in particular, petroleum oils, animal oils, vegetable oils or synthetic oils. Thus, the possible oils can be, for example, peanut oil, soybean oil, mineral oils, sesame oil and the like. Suitable pharmaceutical excipients are selected from the group consisting of starch, cellulose, tallow, glucose, lactose, sucrose, gelatin, malt, rice, flour, chalk, silica gel, magnesium stearate, sodium stearate, glycerol monostearate, sodium chloride, dried skimmed milk, glycerol, propylene glycol, water, ethanol and others.

Suitable buffers, in particular, isotonic buffers, can contain water, sodium chloride, phosphate, citrate, succinate, acetate, adipate, benzoate, lactate, maleate, phosphate, tartrate, borate, trihydroxymethylaminomethane, glycine, histidine and other organic acids or their salts.

Furthermore, the composition, in particular a pharmaceutically tolerable carrier material occurring therein, can contain one or more stabilizers, reducing compounds, antioxidants and/or complexing compounds. The use of buffers, stabilizers, reducing compounds, antioxidants and complexing compounds for the production of pharmaceutical compositions is adequately known to those skilled in the art (Wang et al., “Review of Excipients and pHs for Parenteral Products Used in the United States.” J. Parent Drug Assn., 34(6): 452-462(1980); Wang et al., “Parenteral Formulations of Proteins and Peptides: Stability and Stabilizers,” J. Parent. Sci. and Tech., 42: S4-S26 (Supplement in 1988); Lachman, et al., “Antioxidants and Chelating Agents as Stabilizers in Liquid Dosage Forms-Part 1,” Drug and Cosmetic Industry, 102 (1): 36-38, 40 and 146-148 (1968); Akers, M. J., “Antioxidants in Pharmaceutical Products”, J. Parent. Sci. and Tech., I 36(5): 222-228 (1988); and Methods in Enzymology, Vol. XXV, Colowick and Kaplan eds., “Reduction of Disulfide Bonds in Proteins with Dithiothreitol”, by Konigsberg, pages 185-188).

Suitable salts, for example, are sodium chloride, dextrose, mannitol, sucrose, trehalose and the like. If the composition contains a liquid carrier material, it is preferred that the carrier material has a pH of between approximately 4.5 and 8.5. If the composition, however, contains a pulverulent carrier material, then it is likewise preferred if the carrier material has a pH in a physiologically tolerable and thus nontoxic range.

For an exact volumetric dosage of the composition, a dilution of the antidepressant with a pharmaceutically inactive excipient can be necessary to obtain dosable quantity units which fulfill the requirements of dosing accuracy. If necessary, a dilution can be chosen such that the amount of the composition to be administered, which is preferably administered by an inhaler, contains exactly the desired dose of the antidepressant.

The pharmaceutical composition may be designed for parenteral administration. Possible parenteral administration forms are basically all administration forms with circumvention of the gastrointestinal tract known to the person those skilled in the art. For example, the composition can be designed for intravenous and/or intraarterial administration.

Particularly preferably, the composition is designed for intrapulmonary, preferably inhalative, administration. It can be demonstrated with particular advantage that for an intrapulmonary administration of the composition, smaller amounts or doses of the antidepressant are necessary than with a systemic administration of the composition to achieve a satisfactory prophylactic and/or therapeutic effect. This applies especially with respect to a reduction of the activity of acid sphingomyelinase, a normalization of the cellular ceramide concentration, a reduction of inflammatory reactions, and a normalization of the susceptibility to infection by pathogens. Thus, in the case of an intrapulmonary administration, the antidepressant can be used in a dosage which corresponds to approximately 1/10 to 1/1000 of the dosage as is typically used in a systemic, for example, an oral or intravenous, administration. In other words, it is preferred if the antidepressant is present in the composition in a dose that corresponds to 1/10 to 1/1000 of a systemically, in particularly orally or intravenously, administered dose of the antidepressant. Preferably, the composition contains a dose of the antidepressant of between 0.01 and 20 mg, in particular, 0.1 and 10 mg. In the case of amitriptyline, for example, a dose of between 0.1 and 10 mg is appropriate. In this way, undesired side effects of antidepressants, for example, fatigue and atonia, can be largely avoided. It can be provided for the antidepressant to be supplied in an inhalative administration of the composition in a dose of between 0.01 and 20 mg/day, in particular, 0.1 and 10 mg/day. A further advantage consists in the fact that no systemic effect of the antidepressant takes place in the case of an intrapulmonary administration of the composition. In other words, on intrapulmonary administration the antidepressant displays its effect exclusively in the lung tissue and not also in other tissues.

The antidepressant itself can be selected from the group consisting of serotonin reuptake inhibitors, noradrenaline reuptake inhibitors, dopamine reuptake inhibitors, serotonin/noradrenaline reuptake inhibitors and other monoaminergic antidepressants. The other monoaminergic antidepressants can, in particular, be noradrenergic antidepressants with inhibition of presynaptic alpha-2 receptors, noradrenergic and specifically serotonergic antidepressants, dual-synaptic antidepressants, serotonin (%-HT₂) antagonist and reuptake inhibitors, serotonin reuptake potentiators and/or monoamine oxidase inhibitors.

The antidepressant may be a tricyclic and/or tetracyclic antidepressant. Preferably the antidepressant is a tricyclic antidepressant. Thus, the antidepressant can be a dibenzazepine derivative, in particular a derivative of 10,11-dihydro-5H-dibenz-[b,f]azepine. Suitable anti-depressants are anti-depressants of the amitriptyline type, imipramine type and/or desipramine type.

The antidepressant may be selected from the group consisting of imipramine, amitriptyline, amitriptyline oxide, clomipramine, desipramine, trimipramine, chlorprothixene, fluoxetine, amlodipine, sertraline, lofepramine, nortriptyline, dibenzepin, opipramol, maprotiline, doxepin, mianserin, mirtazapine, dibenzepin, fluvoxamine, citalopram, escitalopram, paroxetine, reboxetine, viloxazine, amineptine, nomifensine, medifoxamine, methylphenidate, venlafaxine, duloxetine, milnacipran, bupropine, trazodone, nefazodone, tianeptene, moclobemide, tranylcypromine, benztropine, chlomiphene, cyproheptadine, cyclobenzaprine, promazine, perhexiline, derivatives thereof and combinations thereof. Particularly preferably, the antidepressant is selected from the group consisting of imipramine, amitriptyline, desipramine, trimipramine, chlorpro-thixene, fluoxetine, amlodipine, sertraline, derivatives thereof and combinations thereof. It can, in particular, be provided for the antidepressant to be present in the form of a physiologically tolerable salt, in particular, in the form of a hydrochloride.

In addition to the antidepressant the pharmaceutical composition may contain at least one further therapeutic active ingredient. The active ingredient can, in particular, be an antibody. The antibodies are usually antibodies which are directed against the acid sphingomyelinase.

As already mentioned, the composition is, in particular, suitable for the prophylaxis or symptomatic treatment of infections or infectious diseases which occur in cystic fibrosis. The infections or infectious diseases can be caused at least by one pathogen from the group consisting of Pseudomonas aeruginosa, Burkholderia cepacia, Staphylococcus aureus and Haemophilus influenzae. The composition is moreover suitable for the prophylaxis and/or therapy of a chronic inflammation in cystic fibrosis.

The composition is present in an advantageous form in sterilized form. For sterilization of the composition, basically all sterilization processes known to those skilled in the art for the sterilization of pharmaceutical compositions, in particular pharmaceutical formulations, are suitable.

Preferably, the pharmaceutical composition is allocated an administration aid, in particular, an inhaler. Preferably, the pharmaceutical composition is contained in an administration aid. The administration aid can be, for example, a spray device, as is customarily obtainable in pharmacies. The administration aid itself with particular advantage contains a mouthpiece, a nebulizer and a pump.

The composition is further preferably present as an aerosol. Preferably, the composition contains particles, in particular aerosol particles, with a diameter, in particular, an aero-dynamic diameter of between 0.1 and 12 μm, in particular 1 and 12 μm, preferably 2 and 12 μm. An aerodynamic diameter is to be understood as the diameter of a particle with a standardized density, for example, with a standardized density of 1 g/cm³, which in air under standard atmospheric conditions has the same settling velocity as the particle itself.

The composition also relates to an aerosolized or sprayed pharmaceutical composition comprising particles with a diameter, in particular, aerodynamic diameter of between 0.1 and 12 where the particles contain at least one pharmaceutically tolerable carrier material and at least one antidepressant. With respect to further details and features, in particular with respect to the antidepressant, the carrier material and the particles, reference is made completely to the preceding description.

In addition, an administration aid, in particular an inhaler which contains the composition, is also included. With respect to further features and details reference is likewise made to the preceding description.

Furthermore, we provide a kit for the prophylaxis and/or symptomatic treatment of cystic fibrosis, in particular for the prophylaxis and/or treatment of infections and/or infectious diseases occurring in cystic fibrosis, where the kit comprises at least one antidepressant and at least one dispersant. The kit can contain a buffer solution, for example, as a further component. Preferably, the antidepressant and the dispersant are contained together in one container. The container can, in particular, be designed as an administration aid for the administration of the antidepressant and the dispersant. It can furthermore be provided for the antidepressant and the dispersant to be present essentially separate from one another in the kit. The antidepressant can be present in powder or tablet form. With respect to further features and details of the kit, in particular of the antidepressant, dispersant and the administration aid, reference is made to the previous description.

Moreover, we also provide a kit comprising:
a device for aerosolizing or spraying a pharmaceutical composition and a cartridge which is connected to the device or attached to the device, and
an aerosolizable or sprayable, inhalable pharmaceutical composition, comprising at least one dispersant and/or at least one pharmaceutically tolerable carrier material and at least one antidepressant.

The device for aerosolizing or spraying the composition can be designed such that an overdose is avoided. This can be done, for example, by electronic monitoring of the dosage amounts to be administered. If the number of doses provided for is achieved, suitable turning off or closing or locking elements can be activated, whereby the administration of the composition is stopped or at least interrupted for a certain time. Moreover, the device for aerosolizing the composition can be programmable, for example, to determine the frequency of administration. Moreover, the device, if desired, can contain a plurality of components which make possible the release of the composition. For example, the device can contain components which allow the timing of spraying relative to inhalation to be controlled. Furthermore, the device can also contain components which supply the patient a feedback signal about the rate and/or the volume of the inhalation. Furthermore, components can be provided which avoid an excessive administration, prevent use by unauthorized persons and/or record the dosage course. In addition, the device can also be designed such that it allows a patient to use only a certain dose for a certain time, and then indicates to the patient that he should use another dose form. For this, the device can be programmed such that the patient begins, for example, with a relatively high dose, which can be administered relatively rapidly, and subsequently the device is only activated if a second, lower dose is chosen. Moreover, the device can be patient- or physician-specifically programmable. The device can also be programmable such that it releases larger or smaller amounts of the pharmaceutical composition and, in particular, forms an aerosol at different rates. Although per se any device can be used which conveys the necessary amounts of the composition to the lungs of a patient, a device with the name Aradigm AerxEssence® is preferred. A suitable device, which is preferably an inhaler, in particular, an electronic inhaler, is described in U.S. Pat. No. 5,718,222 with the title “Disposable package for use in aerosolized delivery of drugs.” Thus, the composition can be administered with the aid of the inhaler described in U.S. Pat. No. 5,718,222, the subject matter of which is incorporated herein by reference.

The device can contain one or more discharge openings for the composition to be aerosolized or atomized. The discharge opening or openings can, in particular, be openings or pores of a porous membrane, through which the composition is forced out in the course of its administration to produce an aerosol. The openings or pores here can all have a uniform size and, in particular, uniform distances to one another. However, it is just as possible that the openings or pores have different sizes and, in particular, are arranged irregularly on the membrane. If the size of the openings varies, the size of particles which are formed in the course of the aerosol formation also varies. Generally, it can be preferable if the discharge openings have a diameter of between approximately 0.25 and 6 μm. In this way, particles with a diameter of approximately 0.5 to 12 μm can be produced, which are particularly preferred with regard to inhalative applications. If the discharge openings have a diameter in the range from 0.25 to 1 μm, an aerosol can be produced thereby which has particles with a diameter in the range from 0.5 μm to 2 μm. This particle diameter is particularly advantageous for administration of the composition, in particular of the antidepressant, to the alveolar duct and to the air sacs (alveoli). Discharge openings with a diameter of approximately 1 to 2 μm can produce particles with a diameter of approximately 2 to 4 μm. These particle diameters are of particular advantage for administration of the composition, in particular of the antidepressant, above the alveolar duct but below the small bronchi. Discharge openings with a diameter of 2 to 4 μm, on the other hand, can produce particles with a diameter of 4 to 8 μm. These particle diameters, on the other hand, with particular advantage allow administration of the composition, in particular of the antidepressant, in the respiratory tract ascending from the small bronchi. A suitable device, for example, is described in U.S. Pat. No. 5,906,202, the subject matter of which is incorporated herein by reference.

A further aspect relates to a cartridge, in particular for use in the intrapulmonary release of active ingredient, comprising:
a container with a nozzle opening, and
an aerosolizable or sprayable, inhalable pharmaceutical composition, comprising at least one dispersant and/or at least one pharmaceutically tolerable carrier material and at least one antidepressant.

Containers which can be used can basically have different forms. For example, the containers can be single-dose packs, for example, blister packs, which preferably contain a liquid and sterile composition. The containers can furthermore have at least a volume of approximately 0.035 to 0.095 cm³, in particular 0.045 to 0.070 cm³. It can moreover be possible that the container is a capsule which contains a dry powder, comprising an antidepressant.

Moreover, we also provide a method for the prophylaxis and/or symptomatic treatment of cystic fibrosis, in particular, for the prophylaxis and/or treatment of infections and/or infectious diseases occurring in cystic fibrosis, where the composition, in particular in a therapeutically efficacious amount, is administered to a patient. The patient can be a human being or an animal, for example, horse, cattle, pig, monkey, rat, mouse or hamster. Preferably, the method, however, is used in a human being. The method is basically equally suitable for male and female patients. Children can also be treated with the aid of the method.

Preferably, the composition is administered intrapulmonarily, preferably inhalatively. For intrapulmonary, preferably inhalative, administration, generally an administration aid suitable for this, in particular an inhaler, is used. Administration aid used can be, for example, a spray device. Preferably, the composition is atomized or aerosolized for inhalative administration.

We furthermore provide a method for the inhibition of acid sphingomyelinase in the lungs of a patient comprising:
aerosolizing or atomizing (spraying) a pharmaceutical composition, comprising at least one dispersant and/or at least one pharmaceutically tolerable carrier material and at least one antidepressant, and
inhaling the aerosolized or atomized (sprayed) composition into the lungs of the patient.

We moreover provide a method for reducing the ceramide concentration in the lungs of a patient comprising:
aerosolizing or atomizing (spraying) a pharmaceutical composition, comprising at least one dispersant and/or at least one pharmaceutically tolerable carrier material and at least one antidepressant, and
inhaling the aerosolized or atomized (sprayed) composition into the lungs of the patient.

Finally, we provide a method for the symptomatic treatment of cystic fibrosis, in particular for the treatment of infections and/or infectious diseases occurring in cystic fibrosis, comprising:
diagnosing cystic fibrosis in a patient,
aerosolizing or atomizing (spraying) a pharmaceutical composition, comprising at least one antidepressant and preferably at least one dispersant and/or at least one pharmaceutically tolerable carrier material,
inhaling the aerosolized or atomized (sprayed) composition into the lungs of the patient and
allowing an adequate amount of particles, comprising at least one antidepressant, to deposit in the lungs of the patient to treat the cystic fibrosis.

The composition is aerosolized or sprayed with particular advantage such that particles are formed which can be inhaled into a lung tissue without difficulty without the particles being exhaled again. Preferably, the aerosolized pharmaceutical composition in the method contains particles with a diameter, in particular aerodynamic diameter, between 0.1 and 12 μm, in particular 1 and 12 μm, preferably 2 and 12 μm.

The antidepressant is preferably administered in a dose that corresponds to 1/10 to 1/1000 of a systemically, in particular orally or intravenously, administered dose of the antidepressant. Particularly preferably, the antidepressant is administered in a daily dose of between 0.01 and 20 mg, in particular 0.1 and 10 mg.

With respect to further features and details of the method, reference is made completely to the previous description.

We also provide for the use of a least one antidepressant for the production of a pharmaceutical composition or of a medicament or pharmaceutical for the prophylaxis and/or symptomatic treatment of cystic fibrosis, in particular for the prophylaxis and/or treatment of infections and/or infectious diseases occurring in cystic fibrosis. With respect to further features and details for this, in particular with respect to suitable antidepressants, reference is likewise made completely to the previous description.

A further aspect relates to the use of the composition for the production of a medicament or drug for the prophylaxis and/or symptomatic treatment of cystic fibrosis, in particular for the prophylaxis, and/or treatment of infections and/or infectious diseases occurring in cystic fibrosis. With respect to further features and details for this, reference is likewise made completely to the previous description.

The features mentioned ensue from the following description of examples in connection with the subclaims and figures. The individual features can in each case be realized separately or together in combination with one another. The figures are hereby made part of the description by express reference.

### EXAMPLES

### 1.1 Cftr-Deficient Mice

The Cftr-deficient mice were originally obtained from Jackson Laboratories (Bar Harbor, Me., USA) and then self-raised at Cycnad. The mice used were deficient with respect to Cftr. This is the mouse equivalent to human CFTR. The experimental animals used, however, expressed human CFTR in the intestine. This was achieved under the control of a promoter for fatty acid-binding protein (FABP). This promoter mediates a specifically intestinal expression of CFTR. The mice used had a mixed genetic background, consisting of C57BL/6, FVB/N and 129.

For the production of further Cftr-knockout mice, genetically homogeneous mice with respect to B6.129P2(CF/3)-Cftr^{TgH(neoim)Hgu} (abbreviated: Cftr^{MHH}) were used. The CF strain CF/3-Cftr^{TgH(neoim)Hgu} produced by inbreeding was raised by a strict pairing of brother and sister animals, starting from a mutated mouse of the original type Cftr^{TgH(neoim)H}. The latter was produced by “insertion mutagenesis” in the Cftr-1 Exon 10. The mouse produced small amounts of Cftr.

In addition, a genetically homogeneous strain of the type Cftr^{MHH} was produced by performing a back-crossing of the target mutation in the background B6 produced by inbreeding. Here, genetically identical B6 mice were used as control animals. The hygienic state of the experimental animals was checked regularly according to the recommendations of FELASA (Federation of European Laboratory Animal Science Associations) issued in 2002.

### 1.2 Inhalation

The inhalation was carried out with the aid of an inhaler (Pariboy® SX). The liquid reservoir in this inhaler was connected to an air nozzle, whereby it was possible to produce a fine aerosol which was inhaled through a mask by the mice. For this, the noses of the experimental animals were pressed manually into the opening of the mask for 10 minutes. This permitted excellent control of the inhalation process. All mice were subjected to an inhalation process 3 times at an interval of 12 hours.

### 1.3 The Determination of the Activity of Acid Sphingomyelinase

The lungs were shock-frozen, homogenized under liquid nitrogen and subsequently transferred to a buffer of 250 mM sodium acetate (pH 5.0), 1.3 mM EDTA and 1% NP40. The homogenizate was then subjected to ultrasound treatment (ultrasonic processor tip sonicator) a total of three times. Subsequently, an aliquot of the samples was diluted with 250 mM sodium acetate (pH 5.0), 1.3 mM EDTA 0.1% NP40 and incubated with 0.05 μCi of [¹⁴C]sphingomyelin per sample (52 mCi/mmol; MP Bio-medicals Irvine Calif., USA). The radioactive sample of [¹⁴C]sphingomyelin was dried, resuspended in 250 mM sodium acetate (pH 5.0), 1.3 mM EDTA and 0.1% NP40 and subsequently subjected to ultrasound treatment for 10 minutes, before it was added to the homogenized samples. The samples were incubated for 30 minutes in total at about 37° C. Subsequently, the enzymatic reaction was ended by extraction (extraction with a solvent mixture of chloroform and methanol in the volume ratio chloroform/methanol 2:1). The samples were then centrifuged and an aliquot of the aqueous phase was in each case subjected to scintillation counting to determine the release of [¹⁴C]phosphorylcholine from [¹⁴C] sphingomyelin.

### 1.4 Measurement of the Ceramide Concentration

After killing the experimental animals, the lungs were removed, homogenized under liquid nitrogen and added to a mixture of chloroform, methanol and 1N hydrochloric acid (100:100:1, v/v/v). After addition of 200 μl of H₂O, the samples were centrifuged for 5 minutes (14,000 rpm, rotations per minute). The lower phase was then collected, dried and subjected to an alkaline hydrolysis of diacylglycerol in 0.1 N methanolic potassium hydroxide solution at 37° C. for 60 minutes. The samples were extracted again, and the lower phase was dried and subsequently resuspended in 20 μl of a detergent solution (7.5% (w/v) of n-octylglucopyranoside, 5 mM cardiolipin in 1 mM diethylenetriaminepentaacetic acid (DTPA)). After an ultrasound treatment (10 minutes), the samples were added to 70 μl of a reaction mixture which contained 10 μl of diacylglycerol kinase (GE Healthcare Europe, Munich, Germany), 0.1 M imidazole/HCl (pH 6.6), 0.2 mM diethylenetriaminepentaacetic acid (pH 6.6), 70 mM sodium chloride, 17 mM magnesium chloride, 1.4 mM EGTA, 2 mM DTT, 1 μM ATP and 10 μCi of [³²P]γATP. The kinase reaction was carried out for 30 minutes at room temperature and subsequently ended by addition of 1 ml of chloroform:methanol:1 N HCl (100:100:1, v/v/v), 170 μl of buffered saline solution (135 mM sodium chloride, 1.5 mM calcium chloride, 0.5 mM magnesium chloride, 5.6 mM glucose, 10 mM HEPES, pH 7.2) and 30 μl of a 100 mM EDTA solution. The samples were then vortexed and the resulting phases were separated. The lower phase was collected, dried, dissolved in 20 μl of a chloroform/methanol mixture (1:1, v/v) and separated by thin-layer chromatography (silica G 60 TLC plates, chloroform:methanol:acetic acid (65:15:5, v/v/v)). The thin-layer chromatography plates were then in each case covered with an X-ray film. The ceramide spots were identified by means of co-migration of a C₁₆-ceramide standard. The incorporation of [³²P] in ceramide was quantified by means of liquid scintillation counting, since the spots had disappeared from the plates. The amounts of ceramide were determined by comparison with a standard curve using C₁₆-ceramide as substrate.

### 1.5 Infection

The bacteria were cultured for 14 to 14.5 hours on TSA agar plates (Becton Dickinson). Fresh plates were used for at most 2 weeks for this. The bacteria were then transferred to Erlenmeyer flasks in 40 ml of a prewarmed and sterile buffer (TSB, Becton Dickinson). The optical density (OD) or extinction was adjusted to 0.225 (corresponds to 5.5×10⁸ CFU/ml) and the bacteria were incubated for 60 minutes at 37° C. with shaking at 125 rpm. The quality of the agar plates and of TSB and the shaking speed were crucial for the obtainment of reproducible results. In this way, it was possible to obtain a Pseudomonas aeruginosa population exactly in the early log phase (the bacteria replicated approximately by 10-fold within an incubation time of 60 minutes). The fact that the bacteria were bacteria of the early log phase was extremely important to obtain reproducible conditions for the subsequent infection with the experimental animals. The bacteria were centrifuged at 2800 rpm (1600×g) in a 50 ml tube. The supernatant was then carefully removed and the bacteria were resuspended in 10 ml of RPMI-1640, supplemented with 10 mM HEPES (pH 7.3). The suspension was transferred to a 15 ml tube and subsequently washed twice. Finally, the bacteria were resuspended in prewarmed HEPES/salt (HS; 132 mM NaCl, 20 mM HEPES (pH 7.4), 5 mM KCl, 1 mM CaCl₂, 0.7 mM MgCl₂, 0.8 mM MgSO₄) and the bacterial optical density was determined. The bacteria were then diluted with prewarmed H/S to a concentration of 10⁸ CFU/20 μl . The mice were then infected within the next 10 minutes. For this purpose, a syringe with a volume of 1 ml and a 30 gauge needle was used. The mice were anesthetized in ether for 10 to 15 seconds. Following this, the needle was immediately inserted 2 mm into the noses of the mice and the bacteria were carefully injected. Between the first and the last mouse which were infected, a time interval of at most 10 minutes was allowed to pass to avoid variations in the formation of viable bacterial colonies in this way. Usually, not more than 10 mice were infected per experiment. Moreover, never more than 1 mouse per experimental group was infected. The mice were killed 2 hours after the administration of Pseudomonas aeruginosa. Subsequently, the number of bacteria in the lung was measured as a measure of the sensitivity of the mice to developing a Pseudomonas aeruginosa infection. For this, the lung was removed, mechanically homogenized, intracellular bacteria were released by a 10-minute treatment with 5 mg/ml of saponin at 37° C., the samples were washed with 10 ml of PBS (phosphate buffered saline), the pellets were taken up in PBS and aliquots thereof were plated out on TSA agar plates. The bacterial colonies were counted after 15 hours growth at 37° C. as a measure of the number of pulmonary bacteria and thus as a measure of an infection.

### 1.6 Measurement of the Secretion of Interleukin-1β

To determine the concentration of interleukin-1β, the lungs were removed 60 minutes after the last inhalation, homogenized under liquid nitrogen and lyzed for 10 minutes in 125 mM NaCl, 25 mM tris HCl (pH 7.4), 10 mM EDTA, 10 mM sodium pyrophosphate, 3% NP40, 10 μg/micrograms of aprotinin and 10 μg/micrograms of leupeptin. An aliquot was then analyzed for interleukin-1β. For this, a commercially obtainable ELISA for interleukin-1β was used. The assay was carried out here exactly according to the protocol of the supplier.

### Experiments

### 2.1 Determination of the Influence of Various Anti-Depressants on the Activity of Acid Sphingomyelinase in Pulmonary Cells

Wild-type mice and Cftr-deficient mice (Cftr-knockout mice) inhaled a solution of 4 mg/l of amitriptyline, 12.5 mg/l of trimipramine, 9 mg/l of desipramine, 8 mg/l of chlorprothixene, 4.5 mg/l of fluoxetine, 5 mg/l of amlodipine and 35 mg/l of sertraline. All substances were dissolved in water or DMSO for this and subsequently diluted at least 1:100 (for DMSO: at least 1:1000) in 0.9% sodium chloride solution. The mice inhaled the solutions for 10 minutes. An inhaler (Pariboy®) was used for the inhalation. In the course of the inhalation time, approximately 2 ml of the solutions administered were atomized. The inhalation was carried out a total of three times at intervals of 12 hours. The lungs were removed 60 minutes after the last inhalation, and the activity of the acid sphingomyelinase was determined in the lung extracts.

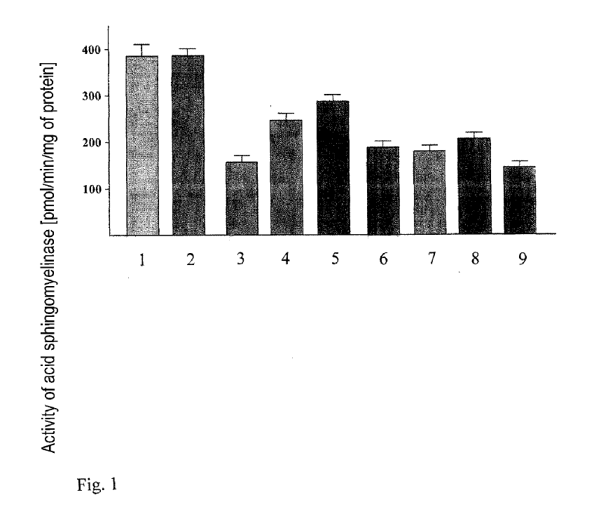

The results are presented graphically in FIG. 1. The activity of the acid sphingomyelinase [pmol/min/mg of protein] is shown here on the ordinate. The Cftr-deficient mice treated with the abovementioned antidepressant solutions in comparison to untreated Cftr-deficient mice and untreated wild-type mice are shown on the abscissa. The figures on the abscissa denote:
1: Cftr-deficient mice, untreated;
2: Wild-type mice, untreated;
3: Cftr-deficient mice after inhalation of an amitriptyline solution (4 mg/l);
4: Cftr-deficient mice after inhalation of a trimipramine solution (12.5 mg/l);
5: Cftr-deficient mice after inhalation of a desipramine solution (9 mg/l);
6: Cftr-deficient mice after inhalation of an amlodipine solution (5 mg/l);
7: Cftr-deficient mice after inhalation of a sertraline solution (35 mg/l);
8: Cftr-deficient mice after inhalation of a fluoxetine solution (4.5 mg/l);
9: Cftr-deficient mice after inhalation of a chlorprothixene solution (8 mg/l).

The results presented graphically in FIG. 1 show that in the treated Cftr-deficient mice it was possible to achieve a significant reduction of the activity of the acid sphingomyelinase.

### 2.2 Determination of the Influence of Various Anti-Depressants on the Pulmonary Ceramide Concentration

Wild-type mice and Cftr-deficient mice were inhaled with a solution of 4 mg/l of amitriptyline, 12.5 mg/l of trimipramine, 9 mg/l of desipramine, 8 mg/l of chlorprothixene, 4.5 mg/l of fluoxetine, 5 mg/l of amlodipine and 35 mg/l of sertraline. The substances were dissolved in water or DMSO for this and subsequently diluted as above in 0.9% sodium chloride solution. The mice inhaled the abovementioned antidepressant solutions through an inhaler suitable for this (Pariboy®). Approximately 2 ml of the administered solutions were atomized during the inhalation time. The inhalation was carried out a total of three times at intervals of 12 hours. The lungs were removed from the experimental animals 60 minutes after the inhalation, and the pulmonary ceramide concentration in the lung extracts was determined.

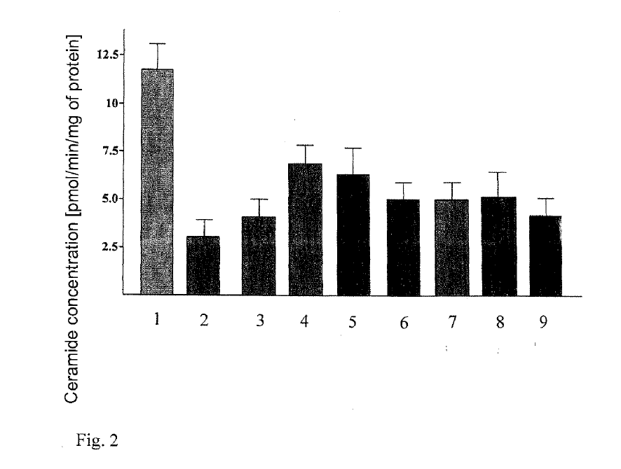

The results are presented graphically in FIG. 2. The pulmonary ceramide concentration [pmol/microgram of protein] is plotted there on the ordinate. The Cftr-deficient mice treated with the individual antidepressant solutions in comparison to untreated Cftr-deficient mice and untreated wild-type mice are shown on the abscissa. The figures on the abscissa denote:
1: Cftr-deficient mice, untreated;
2: Wild-type mice, untreated;
3: Cftr-deficient mice after inhalation of an amitriptyline solution (4 mg/l);
4: Cftr-deficient mice after inhalation of a trimipramine solution (12.5 mg/l);
5: Cftr-deficient mice after inhalation of a desipramine solution (9 mg/l);
6: Cftr-deficient mice after inhalation of an amlodipine solution (5 mg/l);
7: Cftr-deficient mice after inhalation of a sertraline solution (35 mg/l);
8: Cftr-deficient mice after inhalation of a fluoxetine solution (4.5 mg/l);
9: Cftr-deficient mice after inhalation of a chlorprothixene solution (8 mg/l).

The results presented graphically in FIG. 2 show that it was possible to achieve a substantial normalization of the pulmonary ceramide concentration by an inhalative administration of the abovementioned antidepressant solutions in Cftr-deficient mice (mice that have developed cystic fibrosis).

2.3 Determination of the Influence of Various Anti-Depressants on the Susceptibility to Infection in Relation to Pseudomonas aeruginosa

Wild-type mice and Cftr-deficient mice were inhaled with a solution of 4 mg/l of amitriptyline, 12.5 mg/l of trimipramine, 9 mg/l of desipramine, 8 mg/l of chlorprothixene, 4.5 mg/l of fluoxetine, 5 mg/l of amlodipine and 35 mg/l of sertraline. The substances were dissolved in water or DMSO for this and subsequently diluted as above in 0.9% sodium chloride solution. The mice inhaled the abovementioned antidepressant solutions through an inhaler suitable for this (Pariboy®). Approximately 2 ml of the administered solutions were atomized during the inhalation time. The inhalation was carried out a total of three times at intervals of 12 hours. The lungs were removed from the experimental animals 60 minutes after the inhalation, and the susceptibility to infection of the experimental animals in relation to Pseudomonas aeruginosa was determined.

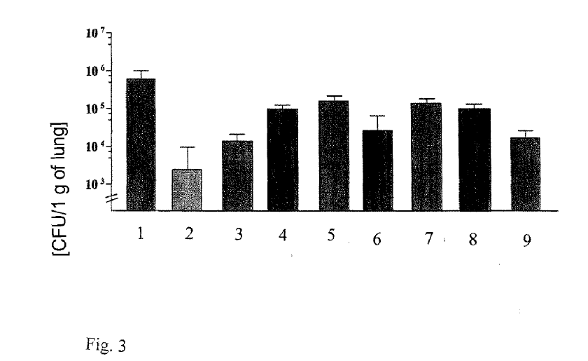

The results obtained here are shown schematically in FIG. 3. The number of colony-forming units (CFU) of Pseudomonas aeruginosa per g of lung tissue is shown there on the ordinate. The Cftr-deficient mice treated with the individual antidepressant solutions in comparison to untreated Cftr-deficient mice and untreated wild-type mice are shown on the abscissa. The figures on the abscissa denote:
1: Cftr-deficient mice, untreated;
2: Wild-type mice, untreated;
3: Cftr-deficient mice after inhalation of an amitriptyline solution (4 mg/l);
4: Cftr-deficient mice after inhalation of a trimipramine solution (12.5 mg/l);
5: Cftr-deficient mice after inhalation of a desipramine solution (9 mg/l);
6: Cftr-deficient mice after inhalation of an amlodipine solution (5 mg/l);
7: Cftr-deficient mice after inhalation of a sertraline solution (35 mg/l);
8: Cftr-deficient mice after inhalation of a fluoxetine solution (4.5 mg/l);
9: Cftr-deficient mice after inhalation of a chlorprothixene solution (8 mg/l).

The results presented graphically in FIG. 3 show that it was possible to achieve a substantial normalization of the susceptibility to infection of the experimental animals in relation to an infection with Pseudomonas aeruginosa on inhalative treatment of Cftr-knockout mice with the abovementioned antidepressant solutions. The experimental animals inhaled the antidepressant solutions here 60 minutes before infection with Pseudomonas aeruginosa. The results presented graphically in FIG. 3 show that the experimental animals are almost resistant to infection by Pseudomonas aeruginosa, in contrast to untreated Cftr-deficient mice. The latter turned out to be susceptible to a high extent to infection by Pseudomonas aeruginosa. This confirms the prophylactic action of the compositions with respect to possible infection by Pseudomonas aeruginosa.

### 2.4 Determination of the Influence of Various Antidepressants on the Secretion of Interleukin-1β

Wild-type mice and Cftr-deficient mice were inhaled with a solution of 4 mg/l of amitriptyline, 12.5 mg/l of trimipramine, 9 mg/l of desipramine, 8 mg/l of chlorprothixene, 4.5 mg/l of fluoxetine, 5 mg/l of amlodipine and 35 mg/l of sertraline. The substances were dissolved in water or DMSO for this and subsequently diluted as above in 0.9% sodium chloride solution. The mice inhaled the abovementioned antidepressant solutions through an inhaler suitable for this (Pariboy®). Approximately 2 ml of the administered solutions were atomized during the inhalation time. The inhalation was carried out a total of three times at intervals of 12 hours. The lungs were removed from the experimental animals 60 minutes after the inhalation, and the concentration of interleukin-1β (IL-1β) in the lung tissue was determined.

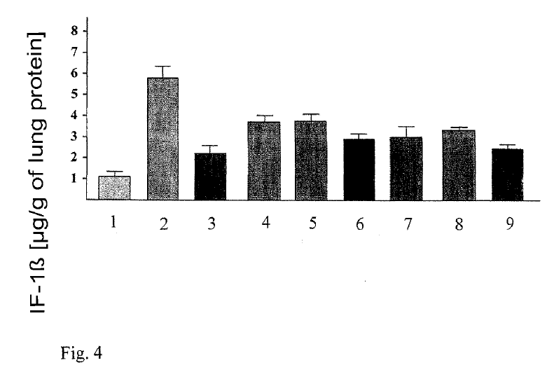

The results are shown graphically in FIG. 4. The concentration of interleukin-1β (μg/g of lung protein) is shown there on the ordinate. Cftr-deficient mice treated with the antidepressant solutions in comparison to untreated Cftr-deficient mice and untreated wild-type mice are shown on the abscissa. The figures on the abscissa denote:
1: Cftr-deficient mice, untreated;
2: Wild-type mice, untreated;
3: Cftr-deficient mice after inhalation of an amitriptyline solution (4 mg/l);
4: Cftr-deficient mice after inhalation of a trimipramine solution (12.5 mg/l);
5: Cftr-deficient mice after inhalation of a desipramine solution (9 mg/l);
6: Cftr-deficient mice after inhalation of an amlodipine solution (5 mg/l);
7: Cftr-deficient mice after inhalation of a sertraline solution (35 mg/l);
8: Cftr-deficient mice after inhalation of a fluoxetine solution (4.5 mg/l);
9: Cftr-deficient mice after inhalation of a chlorprothixene solution (8 mg/l).

### 2.5 Influence of Various Administration Forms by Example of an Amitriptyline Solution

Wild-type mice and Cftr-deficient mice were in each case inhaled 1 ml of an amitriptyline solution (4 mg/l of H₂O) for 20 minutes with the aid of a customary pharmacy inhalation device. The amount of amitriptyline actually administered was therefore about 4 μg. In parallel to this, other wild-type mice and Cftr-deficient mice were in each case administered 100 μl of an amitriptyline solution (2.5 g/l of H₂O), so that the amount of amitriptyline actually administered in these animals was 250 μg. The injections were administered twice daily over 2 days.

In the case of the inhalative treatment, the lungs of the mice were extracted 60 minutes or 12 hours after ending the inhalation, where in the case of the 12-hour experiments the inhalation was repeated after 6 hours. In the case of the mice treated by an injection solution, their lungs were removed 12 hours after the last injection.

The lung extracts were in each case investigated for the acid sphingomyelinase activity, the ceramide concentration and for population by Pseudomonas aeruginosa.

The following results were actually obtained here.

### 2.5.1 Acid Sphingomyelinase Activity

An enzyme activity of 405±19 pmol/min/mg of protein was detected in the lung extracts of untreated wild-type mice. The lung extracts of untreated, Cftr-deficient mice showed an enzyme activity of 375±19 pmol/min/mg of protein.

An activity of 176±18 pmol/min/mg of protein was determined in the lungs of wild-type mice treated by inhalation extracted after 60 min. An enzyme activity of 148±17 pmol/min/mg of protein was measured in the lung extracts of correspondingly treated, Cftr-deficient mice.

The lungs of inhalatively treated wild-type mice extracted after 12 hours showed an enzyme activity of 200±14 pmol/min/mg of protein. The lung extracts of correspondingly treated, Cftr-deficient mice showed an enzyme activity of 167±9 pmol/min/mg of protein.

The lungs of the wild-type mice treated by an injection solution showed an enzyme activity of 168±19 pmol/min/mg of protein. The lung extracts of correspondingly treated, Cftr-deficient mice showed an enzyme activity of 151±17 pmol/min/mg of protein.

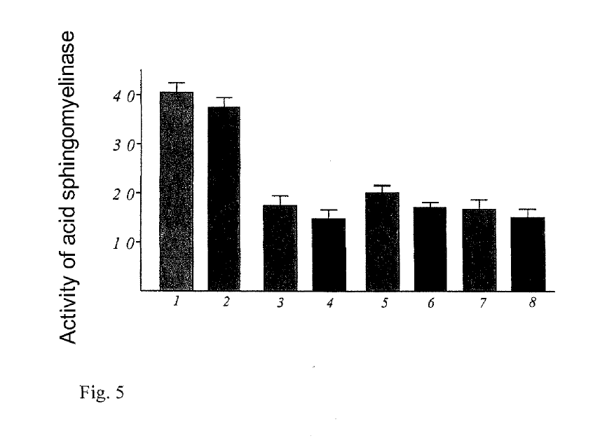

These results are shown schematically in FIG. 5. The activity of the acid sphingomyelinase, measured in pmol/min/mg of protein, is plotted on the ordinate. The wild-type and Cftr-deficient mice and the various administration or presentation forms are listed on the abscissa. The figures on the abscissa denote:
1: Wild-type mice, untreated;
2: Cftr-deficient mice, untreated;
3: Wild-type mice; activity of acid-sphingomyelinase 60 min after inhalation of 1 ml of an amitriptyline solution (4 mg/l);
4: Cftr-deficient mice, activity of acid sphingomyelinase 60 min after inhalation of 1 ml of an amitriptyline solution (4 mg/l);
5: Wild-type mice, activity of acid sphingomyelinase 12 h after inhalation of 1 ml of an amitriptyline solution (4 mg/l);
6: Cftr-deficient mice, activity of acid sphingomyelinase 12 h after inhalation of 1 ml of an amitriptyline solution (4 mg/l);
7: Wild-type mice, activity of acid sphingomyelinase 12 h after intraperitoneal injection of 250 micrograms of amitriptyline;
8: Cftr-deficient mice, activity of acid sphingomyelinase 12 h after intraperitoneal injection of 250 micrograms of amitriptyline.

FIG. 5 makes clear that the composition with particular advantage brings about significant inhibition of acid sphingomyelinase in the presence of cystic fibrosis. The results in particular show that the inhibition of ceramide-releasing enzyme can be between 55 and 66%, based on the cellular total activity of acid sphingomyelinase in a Cftr-deficient mouse. In the case of an inhalative administration of the composition, it is moreover advantageous that this action can even be achieved with low amitriptyline doses (effective administered dose of amitriptyline in inhalative administration was 4 μg). Undesired side effects, which usually occur with increasing amitriptyline doses, can thereby be avoided.

### 2.5.2 Ceramide Concentration:

In the lung extracts of untreated wild-type mice, it was possible to measure a ceramide concentration of 2.3±0.5 pmol/μg of protein. The lung extracts of untreated, Cftr-deficient mice showed a ceramide concentration of 16±1.6 pmol/pg of protein.

In the lungs of wild-type mice extracted after 12 h, a ceramide concentration of 1.6±0.3 pmol/μg of protein was measured. The lung extracts of correspondingly treated, Cftr-deficient mice showed a ceramide concentration of 5.2±0.79 pmol/μg of protein.

The lung extracts of parenterally treated wild-type mice showed a ceramide concentration of 1.5±0.4 pmol/μg of protein. The ceramide concentration in the lung extracts of correspondingly treated, Cftr-deficient mice was 4.9±0.7 pmol/μg of protein.

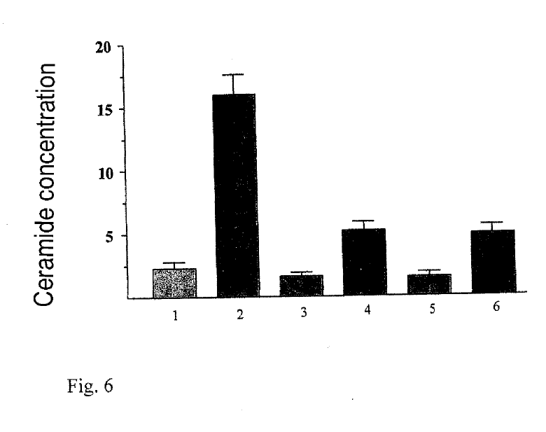

The results listed above are shown schematically in FIG. 6. The ceramide concentration, measured in pmol/μg of lung protein, is indicated on the ordinate. The wild-type and Cftr-deficient mice including the various administration or presentation forms are listed on the abscissa. The figures on the abscissa denote:
1: Wild-type mice, untreated;
2: Cftr-deficient mice, untreated;
3: Wild-type mice, ceramide concentration 12 h after inhalation of 1 ml of an amitriptyline solution (4 mg/l);
4: Cftr-deficient mice, ceramide concentration 12 h after inhalation of 1 ml of an amitriptyline solution (4 mg/l);
5: Wild-type mice, ceramide concentration 12 h after intraperitoneal injection of 250 micrograms of amitriptyline;
6: Cftr-deficient mice, ceramide concentration 12 h after intraperitoneal injection of 250 micrograms of amitriptyline.

It is evident from FIG. 6 that the composition is particularly suitable for bringing about a normalization of the ceramide concentration in the presence of cystic fibrosis. Reference is made to what has already been said under 2.5.1 with respect to further advantages.

2.5.3 Measurement of the Susceptibility to Infection for Pseudomonas aeruginosa

In these experiments, wild-type mice and Cftr-deficient mice were administered 1×10⁸ CFU (colony forming units) of Pseudomonas aeruginosa strain 762 into the nose after double inhalation with in each case 1 ml of an amitriptyline solution (4 mg/l of H₂O). Untreated mice were used as controls. The mice were killed 2 hours after administration. Subsequently, the number of bacteria in the lung was measured as a measure of the sensitivity of the mice of developing a Pseudomonas aeruginosa infection. For this, the lung was removed, mechanically homogenized, intracellular bacteria were released by a 10-minute treatment with 5 mg/ml of saponin at 37° C., the samples were washed with 10 ml of PBS (phosphate buffered saline), the pellets were taken up in PBS and aliquots thereof were plated out on TSA agar plates. The bacterial colonies were counted after 15 hours growth at 37° C. as a measure of the number of pulmonary bacteria and thus as a measure of an infection. The following results were obtained here:

It was possible to measure a bacterial count of 4700±2160 CFU in the lungs of untreated wild-type mice. The removed lungs of untreated, Cftr-deficient mice showed a bacterial count of 6.3×10⁶±1.87×10⁶ CFU. The lungs of wild-type mice treated by inhalation showed a bacterial count of 3600±2892 CFU. In the lungs of inhalatively treated, Cftr-deficient mice, it was possible to determine a bacterial count of 12300±4075 CFU. The lungs of wild-type mice treated by injection solution showed a bacterial count of 2780±1740 CFU. The lungs of correspondingly treated Cftr-deficient mice showed a bacterial count of 11250±2360 CFU.

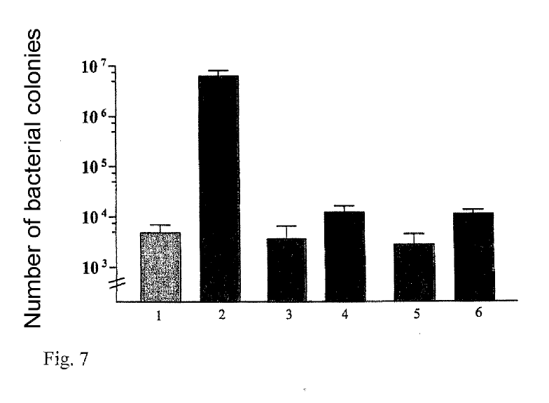

The abovementioned results are shown graphically in FIG. 7. The number of bacteria (CFU, colony forming units), measured in one gram of lung, is plotted there on the ordinate. The untreated or treated wild-type mice or Cftr-deficient mice including the various administration forms are listed on the abscissa. The figures on the abscissa denote:
1: Wild-type mice, bacterial counts in the lung 2 h after intranasal infection, no further treatment;
2: Cftr-deficient mice, bacterial counts in the lung 2 h after intranasal infection, no further treatment;
3: Wild-type mice, bacterial counts in the lung 12 h after inhalation of 1 ml of an amitriptyline solution (4 mg/l) and 2 h after intranasal infection;
4: Cftr-deficient mice, bacterial counts in the lung 12 h after inhalation of 1 ml of an amitriptyline solution (4 mg/l) and 2 h after intranasal infection;
5: Wild-type mice, bacterial counts in the lung 12 h after intraperitoneal injection of 250 micrograms of amitriptyline and 2 h after intranasal infection;
6: Cftr-deficient mice, bacterial counts in the lung 12 h after intraperitoneal injection of 250 micrograms of amitriptyline and 2 h after intranasal infection.

It can be clearly inferred here from FIG. 7 that the administration of the composition leads to a substantial normalization of the susceptibility to infection for Pseudomonas aeruginosa. FIG. 7, in particular, illustrates the suitability of the composition for the prophylaxis and/or therapy of infections or infectious diseases that occur in connection with cystic fibrosis. Reference is likewise made to what has already been said under 2.5.1 with respect to further advantages.

### 2.6 Investigation of a Possible Systemic Effect

The spleen was removed from the experimental mice 60 minutes after the last inhalation. The spleen was then mechanically comminuted. The splenocytes were washed twice in H/S and lyzed in 250 mM sodium acetate (pH 5.0), 1.3 mM EDTA and 1% NP40. The activity of acid sphingomyelinase was then determined as described previously. It turned out here that the inhalative administration of the antidepressants had no effect at all on the activity of the acid sphingomyelinase in the spleen cells (splenocytes) of the experimental animals. This shows that no systemic effects occurred in the case of inhalative administration.

1. A pharmaceutical composition for prophylaxis and/or treatment of infections and/or infectious diseases occurring in cystic fibrosis comprising:
at least one antidepressant; and
at least one selected from the group consisting of a dispersant and
a pharmaceutically tolerable carrier material.

2. The pharmaceutical composition as claimed in claim 1, wherein the antidepressant is dispersed in the dispersant.

3. The pharmaceutical composition as claimed in claim 1, wherein the dispersant is liquid.

4. The pharmaceutical composition as claimed in claim 1, wherein the dispersant is a physiological fluid.

5. The pharmaceutical composition as claimed in claim 1, present in the form of a liquid formulation.

6. The pharmaceutical composition as claimed in claim 1, present in the form of an aqueous solution.

7. The pharmaceutical composition as claimed in claim 1, in the form of an intrapulmonary inhalative.

8. The pharmaceutical composition as claimed in claim 1, wherein the antidepressant is present in the composition in a dose that corresponds to 1/10 to 1/1000 of a systemically administered dose of the antidepressant.

9. The pharmaceutical composition as claimed in claim 1, wherein the composition contains an amount of the antidepressant of between 0.01 and 20 mg.

10. The pharmaceutical composition as claimed in claim 1, wherein the antidepressant is selected from the group consisting of serotonin reuptake inhibitors, noradrenaline reuptake inhibitors, dopamine reuptake inhibitors, serotonin/noradrenaline reuptake inhibitors and other monoaminergic antidepressants.

11. The pharmaceutical composition as claimed in claim 1, wherein the antidepressant is a tri- or tetracyclic antidepressant.

12. The pharmaceutical composition as claimed in claim 1, wherein the antidepressant is derivative of 10,11-dihydro-5H-dibenz-[b,f]azepine.

13. The pharmaceutical composition as claimed in claim 1, wherein the antidepressant is selected from the group consisting of imipramine, amitriptyline, amitriptyline oxide, clomipramine, desipramine, trimipramine, chlorprothixene, fluoxetine, amlodipine, sertraline, lofepramine, nortriptyline, dibenzepin, opipramol, maprotiline, doxepin, mianserin, mirtazapine, dibenzepin, fluvoxamine, citalopram, escitalopram, paroxetine, reboxetine, viloxazine, amineptine, nomifensine, medifoxamine, methylphenidate, venlafaxine, duloxetine, milnacipran, bupropine, trazodone, nefazodone, tianeptene, moclobemide, tranylcypromine, benztropine, chlomiphene, cyproheptadine, cyclobenzaprine, promazine, perhexeline, derivatives thereof and combinations thereof.

14. The pharmaceutical composition as claimed in claim 1, wherein the antidepressant is selected from the group consisting of imipramine, amitriptyline, desipramine, trimipramine, chlorprothixene, fluoxetine, amlodipine, sertraline, derivatives thereof and combinations thereof.

15. The pharmaceutical composition as claimed in claim 1, wherein the composition contains, in addition to the antidepressant, at least one further therapeutic active ingredient, in particular, an antibody.

16. The pharmaceutical composition as claimed in claim 1, wherein the infections or infectious diseases are caused at least by one pathogen from the group consisting of Pseudomonas aeruginosa, Pseudomonas species, Staphylococcus aureus, Burkholderia cepacia and Haemophilus influenzae.

17. (canceled)

18. The pharmaceutical composition as claimed in claim 1, in the form of an aerosol.

19. The pharmaceutical composition as claimed in claim 1, further comprising aerosol particles with a diameter of between 0.1 and 12 μm.

20. An aerosolized pharmaceutical composition, comprising particles with an aerodynamic diameter in a range from about 0.1 to 12 μm, where the particles contain at least one pharmaceutically tolerable, inhalable carrier material and at least one antidepressant.

21. The aerosolized pharmaceutical composition as claimed in claim 20, wherein the particles have a diameter of approximately 1 to 12 μm.

22. The aerosolized pharmaceutical composition as claimed in claim 20, wherein the antidepressant is selected from the group consisting of imipramine, amitriptyline, desipramine, trimipramine, chlorprothixene, fluoxetine, amlodipine, sertraline, derivatives thereof and combinations thereof.

23. An inhaler, comprising a pharmaceutical composition as claimed in claim 1.

24. A kit for prophylaxis and/or treatment of infections and/or infectious diseases occurring in cystic fibrosis, comprising at least one antidepressant, at least one dispersant and/or at least one pharmaceutically tolerable carrier material.

25. A kit comprising:
a device for atomizing a pharmaceutical composition, and
a cartridge which is connected to the device, and an aerosolizable, inhalable pharmaceutical composition, comprising at least one dispersant and/or at least one pharmaceutically tolerable carrier material and at least one antidepressant.

26. A cartridge for use in the intrapulmonary release of active ingredient, comprising:
a container with a nozzle opening, and
an aerosolizable, inhalable pharmaceutical composition, comprising at least one dispersant and/or at least one pharmaceutically tolerable carrier material and at least one antidepressant.

27. A method for inhibiting acid sphingomyelinase in lungs of a patient, comprising:
aerosolizing or atomizing a pharmaceutical composition comprising at least one dispersant and/or at least one pharmaceutically tolerable carrier material and at least one antidepressant, and
inhaling the aerosolized or atomized pharmaceutical composition into the lungs of the patient.

28. A method for reducing ceramide concentration in lungs of a patient, comprising:
aerosolizing or atomizing a pharmaceutical composition, comprising at least one dispersant and/or at least one pharmaceutically tolerable carrier material and at least one antidepressant, and
inhaling the aerosolized or atomized pharmaceutical composition into the lungs of the patient.

29. A method for treating infections and/or infectious diseases occurring in cystic fibrosis comprising:
diagnosing cystic fibrosis in a patient,
aerosolizing or atomizing a pharmaceutical composition comprising at least one antidepressant and at least one dispersant and/or at least one pharmaceutically tolerable carrier material,
inhaling the aerosolized or atomized composition into the lungs of the patient, and
allowing an adequate amount of particles, comprising at least one antidepressant, to deposit in the lungs of the patient in order to treat cystic fibrosis.

30. The method as claimed in claim 27, wherein the antidepressant is selected from the group consisting of imipramine, amitriptyline, desipramine, trimipramine, chlorprothixene, fluoxetine, amlodipine, sertraline, derivatives thereof and combinations thereof.

31. The method as claimed in claim 27, wherein the aerosolized pharmaceutical composition contains particles with a diameter of 0.1 and 12 μm.

32. The method as claimed in claim 27, wherein a daily dose of the antidepressant is administered that corresponds to 1/100 to 1/1000 of an orally or intravenously administered dose of the antidepressant.

33. The method as claimed in claim 27, wherein the antidepressant is administered in a dose of between 0.01 and 20 mg/day.

34. (canceled)

35. The method as claimed in claim 28, wherein the antidepressant is selected from the group consisting of imipramine, amitriptyline, desipramine, trimipramine, chlorprothixene, fluoxetine, amlodipine, sertraline, derivatives thereof and combinations thereof.

36. The method as claimed in claim 29, wherein the antidepressant is selected from the group consisting of imipramine, amitriptyline, desipramine, trimipramine, chlorprothixene, fluoxetine, amlodipine, sertraline, derivatives thereof and combinations thereof.

37. The method as claimed in claim 28, wherein the aerosolized pharmaceutical composition contains particles with a diameter of 0.1 and 12 μm, in particular 1 to 12 μm.

38. The method as claimed in claim 29, wherein the aerosolized pharmaceutical composition contains particles with a diameter of 0.1 and 12 μm.

39. The method as claimed in claim 30, wherein the aerosolized pharmaceutical composition contains particles with a diameter of 0.1 and 12 μm.

40. The method as claimed in claim 28, wherein a daily dose of the antidepressant is administered that corresponds to 1/100 to 1/1000 of an orally or intravenously administered dose of the antidepressant.

41. The method as claimed in claim 29, wherein a daily dose of the antidepressant is administered that corresponds to 1/100 to 1/1000 of an orally or intravenously administered dose of the antidepressant.

42. The method as claimed in claim 30, wherein a daily dose of the antidepressant is administered that corresponds to 1/100 to 1/1000 of an orally or intravenously administered dose of the antidepressant.

43. The method as claimed in claim 31, wherein a daily dose of the antidepressant is administered that corresponds to 1/100 to 1/1000 of an orally or intravenously administered dose of the antidepressant.

44. The method as claimed in claim 28, wherein the antidepressant is administered in a dose of between 0.01 and 20 mg/day.

45. The method as claimed in claim 29, wherein the antidepressant is administered in a dose of between 0.01 and 20 mg/day.

46. The method as claimed in claim 30, wherein the antidepressant is administered in a dose of between 0.01 and 20 mg/day.

47. The method as claimed in claim 31, wherein the antidepressant is administered in a dose of between 0.01 and 20 mg/day.

48. The method as claimed in claim 32, wherein the antidepressant is administered in a dose of between 0.01 and 20 mg/day.
